(19) United States
(12) Patent Application Publication
(10) Pub. No.: US 2026/0106045 A1
(43) Pub. Date: Apr. 16, 2026

(54) PROVIDING AUTOMATED ASSISTANCE IN RESPONDING TO TEXTUAL MESSAGES TO PROFESSIONALS FROM THEIR CLIENTS

(71) Applicant: Providence St. Joseph Health, Renton, WA (US)

(72) Inventors: Maulin Shah, Portland, OR (US); Guilford Parsons, Seattle, WA (US)

(21) Appl. No.: 18/913,457

(22) Filed: Oct. 11, 2024

(51) Int. Cl. G16H 80/00

(52) U.S. Cl. CPC G16H 80/00

(57) ABSTRACT

A facility for analyzing an incoming textual message received from a sender is described. When the message is received, the facility invokes a large language model against it at least once to (1) identify in the message one or more topics for which response would be appropriate and (2) generate candidate response themes for each of the identified topics. For each of the identified topics, the facility causes display of controls that visually identify each of the generated candidate response themes.

### BACKGROUND

Professionals of many types must respond to significant volumes of textual correspondence from their clients. For example, as electronic medical engagement systems become more common, physicians receive and must respond to increasing volumes of electronic textual messages from their patients.

A typical way for providing responses to such patient messages involves a physician reading the message, reviewing relevant portions of the sending patient's medical record, and handwriting or typing a treatment plan for the patient in a compact medical shorthand. The treatment plan is passed to a medical assistant, who translates the treatment plan in shorthand into a typed response in conventional English, which the medical assistant sends to the sending patient.

### BRIEF DESCRIPTION OF THE DRAWINGS

FIG. 1 is a network diagram showing an environment in which the facility operates in some embodiments.

FIG. 2 is a block diagram showing some of the components typically incorporated in at least some of the computer systems and other devices on which the facility operates.

FIG. 3 is a dataflow diagram showing a process performed by the facility in some embodiments to process an incoming message.

FIG. 4 is a display diagram showing a sample display by the facility in some embodiments to process an incoming message.

FIG. 5 is a flow diagram showing a process performed by the facility in some embodiments to process an incoming message.

### DETAILED DESCRIPTION

The inventors have recognized that conventional approaches to responding to incoming textual messages to professionals such as physicians from their clients have significant disadvantages. A first disadvantage of these conventional approaches is that they have low time efficiency, both with respect to the large amount of time that people (e.g., physicians and medical assistants) spend in responding to a single incoming message, and the large amount of time that elapses between when the message is received and its response is sent.

A second disadvantage is that the conventional process leads to individual messages being handled in a fragmented manner, sometimes leading to errors when a person involved in generating the response fails to connect their present work on the message to their earlier work on the message.

In response to recognizing these disadvantages of conventional techniques, the inventors have conceived and reduced to practice a software and/or hardware facility for providing automated assistance in responding to textual messages to professionals from their clients (“the facility”). In particular, the facility automatically proposes possible response elements based upon the contents of a message; when the professional selects certain proposed response elements, the facility automatically generates an outgoing natural language message incorporating the selected elements. In some embodiments, instead of or in addition to selecting automatically proposed response elements, the professional prepares a design for the response in domain shorthand. In some embodiments, the facility automatically generates a response by combining input from the professional with contents of the message. In some embodiments, the facility automatically tunes the automatically generated message to attributes of the patient such as educational level, age, dialect, etc. The professional can revise the outgoing message before it is sent.

In some embodiments, the facility submits a received client message to a large language model (LLM), along with a prompt instructing the LLM to identify topics included in the client message. The facility then submits these identified topics to an LLM along with a prompt instructing it to determine possible response themes for each of the identified topics in a compact shorthand, such as one typically used in the professional's field. The facility displays these determined possible response themes to each topic as buttons, and receives input from the professional selecting certain of the buttons. The facility generates a response strategy by collecting the shorthand representations of the response themes whose buttons were selected, submits this response strategy to an LLM along with a prompt instructing it to generate an outgoing natural language message that is based on the selected responses. The professional can revise this outgoing message before it is sent to the client. In various embodiments, the facility uses automation tools other than an LLM to generate the outgoing message, such as machine learning models of other types, natural language processing, or procedural programs.

In some embodiments, the facility displays additional shorthand responses that can be selected by the professional that relate to the context that presently exists between the professional and the client. For example, the facility may display additional shorthand responses to a physician that relate to recent orders and prescriptions made by the physician for the sending patient, as retrieved from electronic health record (“EHR”) system that records this information.

By operating in some or all of the ways described above, the facility enables professionals to more quickly, accurately, and conveniently respond to client messages. The facility further provides responses to clients that they find more complete, accurate, readable, understandable, and/or actionable.

Additionally, the facility improves the functioning of computer or other hardware, such as by reducing the dynamic display area, processing, storage, and/or data transmission resources needed to perform a certain task, thereby enabling the task or portions of it to be permitted by less capable, capacious, and/or expensive hardware devices, and/or be performed with lesser latency, and/or preserving more of the conserved resources for use in performing other tasks. For example, by obviating the manual entry of the outgoing message, the facility avoids committing any processing resources to operating the user interface into which the outgoing message is manually entered.

Further, for at least some of the domains and scenarios discussed herein, the processes described herein as being performed automatically by a computing system cannot practically be performed in the human mind, for reasons that include that the starting data, intermediate state(s), and ending data are too voluminous and/or poorly organized for human access and processing, and/or are a form not perceivable and/or expressible by the human mind; the involved data manipulation operations and/or subprocesses are too complex, and/or too different from typical human mental operations; required response times are too short to be satisfied by human performance; etc.

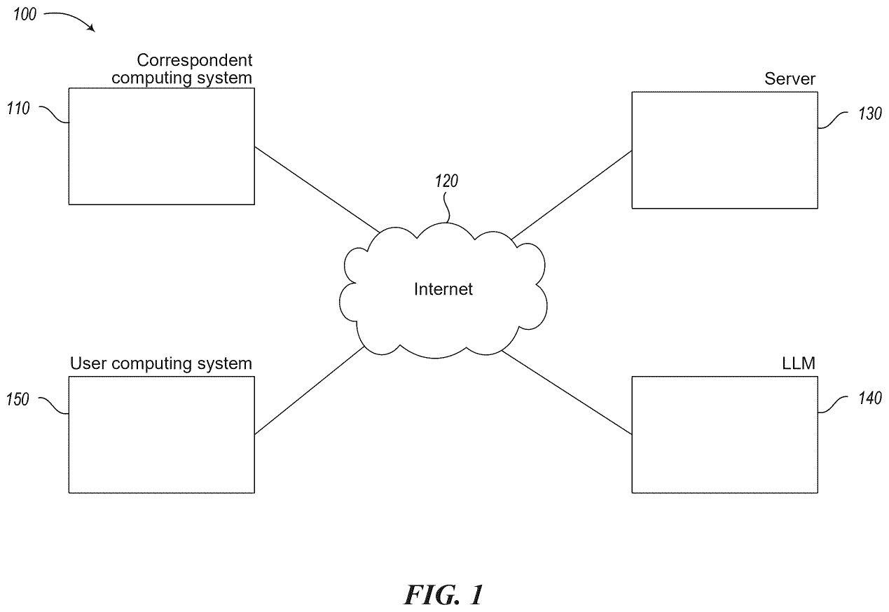

FIG. 1 is a network diagram showing an environment in which the facility operates in some embodiments. In the environment 100, a user-such as a patient of a physician-uses a correspondent computer system 110 to prepare a message to a recipient, such as a physician or other professional. For example, in some embodiments, the sender interacts with a patient-facing interface of an electronic health record (“EHR”) system accessed via the Internet 120 or other network and a server 130. The physician or other professional to whom the incoming message is addressed uses a user computing system 150 to interact with the message, such as on the server 130 via the internet. These interactions permit the addressee user to see the incoming message, plus controls-such as buttons-representing response themes that may be appropriate in light of topics identified by the facility in the incoming message using an LLM 140, or one or more other artificial intelligence mechanisms. When the addressee user selects certain of these buttons, the facility generates a response strategy in a compact shorthand, which the user can edit or simply approve for transformation into a complete outgoing message by the LLM.

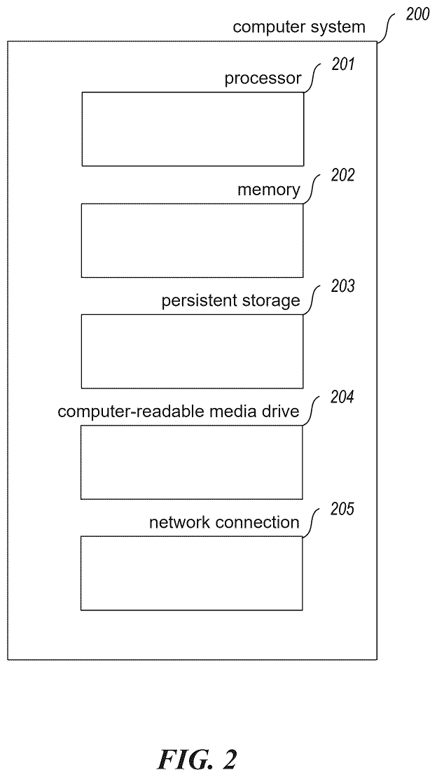

FIG. 2 is a block diagram showing some of the components typically incorporated in at least some of the computer systems and other devices on which the facility operates. In various embodiments, these computer systems and other devices 200 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 201 for executing computer programs and/or training or applying machine learning models, such as a CPU, GPU, TPU, NNP, FPGA, or ASIC; a computer memory 202—such as RAM, SDRAM, ROM, PROM, etc.—for storing programs and data while they are being used, including the facility and associated data, an operating system including a kernel, and device drivers; a persistent storage device 203, such as a hard drive or flash drive for persistently storing programs and data; a computer-readable media drive 204, such as a floppy, CD-ROM, or DVD drive, for reading programs and data stored on a computer-readable medium; and a network connection 205 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. None of the components shown in FIG. 2 and discussed above constitutes a data signal per se. While computer systems configured as described above are typically used to support the operation of the facility, those skilled in the art will appreciate that the facility may be implemented using devices of various types and configurations, and having various components.

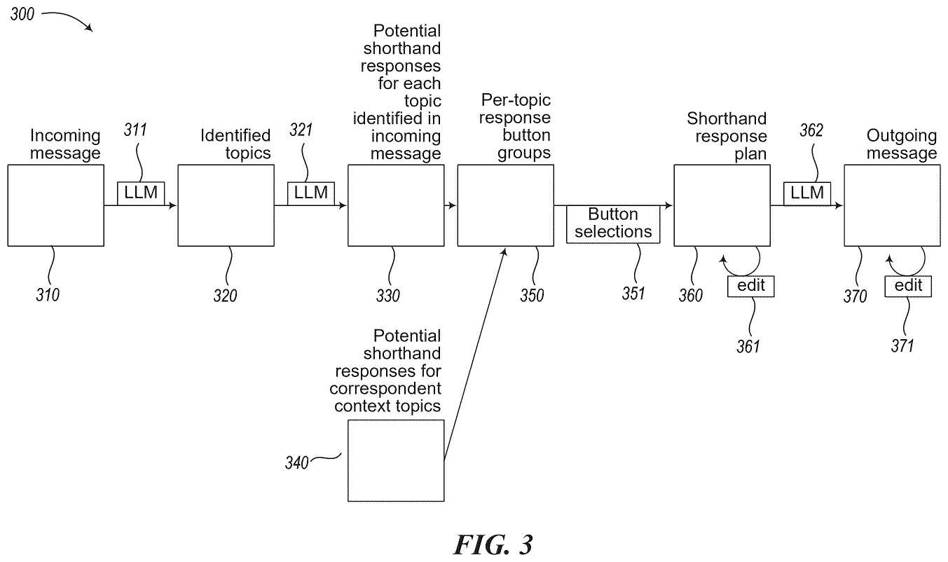

FIG. 3 is a dataflow diagram showing a process performed by the facility in some embodiments to process an incoming message. In the dataflow 300, the facility subjects the incoming message 310 to an LLM 311 to identify topics 340 in the incoming message, and ultimately generate potential shorthand responses 330 for each topic identified in the incoming message. The facility uses these potential shorthand responses to generate a response strategy button group 350 for each topic that is displayed to the addressee user. In some embodiments, the facility also generates potential shorthand responses 340 for relating to context of the correspondent who sent the message; for example, in the case of a patient sending a message to their physician, these context topics can include orders and prescriptions recently made for the patient by the physician. These topics, too, are the subject of response button groups 350. When the facility displays these button groups and the user makes button selections 351 among them, the facility generates a shorthand response plan 360, such as by collecting the shorthand characterizations of the response theme candidates for the selected buttons. The user can edit 361 the response plan to better tailor it to the message that the user wishes to send. In some embodiments, the user directly enters the shorthand response plan, such as by typing it or speaking it. The facility subjects the shorthand response plan to an LLM 362 to generate a draft 370 of the outgoing message, which the user can edit 371 before it is sent to the correspondent who sent the original message.

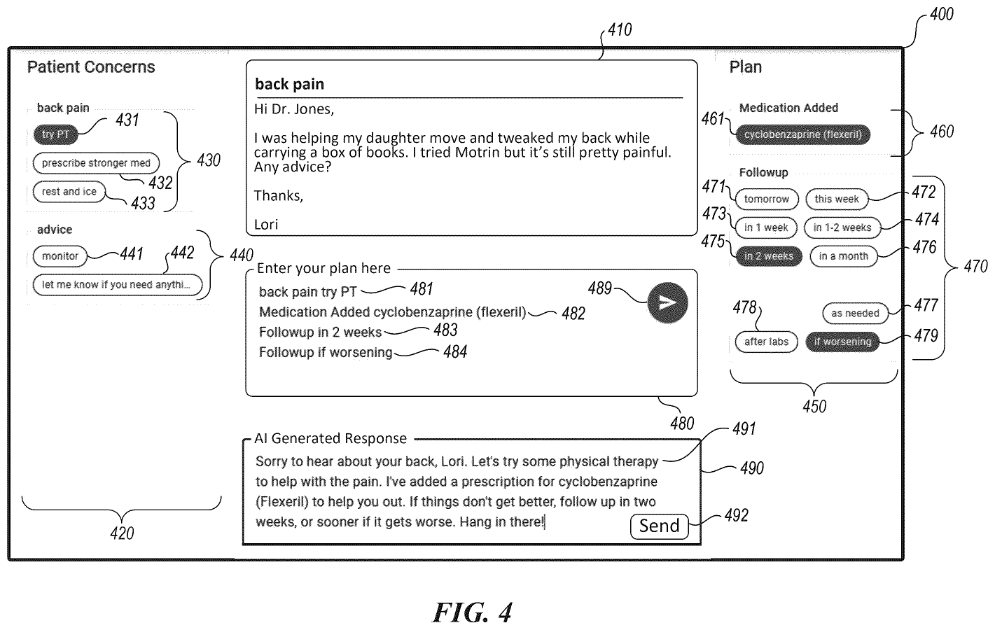

FIG. 4 is a display diagram showing a sample display by the facility in some embodiments to process an incoming message. The display 400 includes incoming message 410, made up of the subject “back pain” and the message body shown beneath it. In this incoming message, the facility has identified two topics, shown in message topic area 420: a back pain topic 430, an advice topic 440. For the back pain topic, the facility has determined three candidate response strategies 431-433, each expressed in compact shorthand. Each of these is displayed as a button that is selectable by the user. For advice topic 440, the facility has similarly identified two candidate response themes 441 and 442, for which it has displayed selectable buttons. Within these topics, the user has selected candidate response theme 431.

The display also includes correspondent context topic area 450, in which the facility displays buttons for further populating the response plan. This includes a medication added topic 460 and a followup topic 470. Within these topics, the user has selected candidate response themes 461, 475, and 479.

The facility has populated response plan 480 automatically based upon the selection of these four buttons. In particular, the facility has added shorthand characterization 481 based upon the selection of button 431 in back pain topic 430; shorthand characterization 482 based upon the selection of button 461 in medication added 460; shorthand characterization 483 based upon the selection of button 471 in followup topic 470; and shorthand characterization 484 based upon the selection of button 479 in followup topic 470. At this point, the user can revise this plan, such as by adding, deleting, or reordering entire shorthand characterizations, and/or changing the specific contents of one or more shorthand characterizations.

After the user is satisfied with the response plan, the user selects control 489 in order to generate a draft outgoing message. The draft outgoing message 491 is shown in window 490. At this point, the user can revise the outgoing message to more closely conform to how the user wishes to interact with the sender of the incoming message. After the user is satisfied with the draft message, the user activates a send control 492 to send the draft message to the sender of the incoming message.

While FIG. 4 shows a display whose formatting, organization, informational density, etc., is best suited to certain types of display devices, those skilled in the art will appreciate that actual displays presented by the facility may differ from those shown, in that they may be optimized for particular other display devices, or have shown visual elements omitted, visual elements not shown included, visual elements reorganized, reformatted, revisualized, or shown at different levels of magnification, etc.

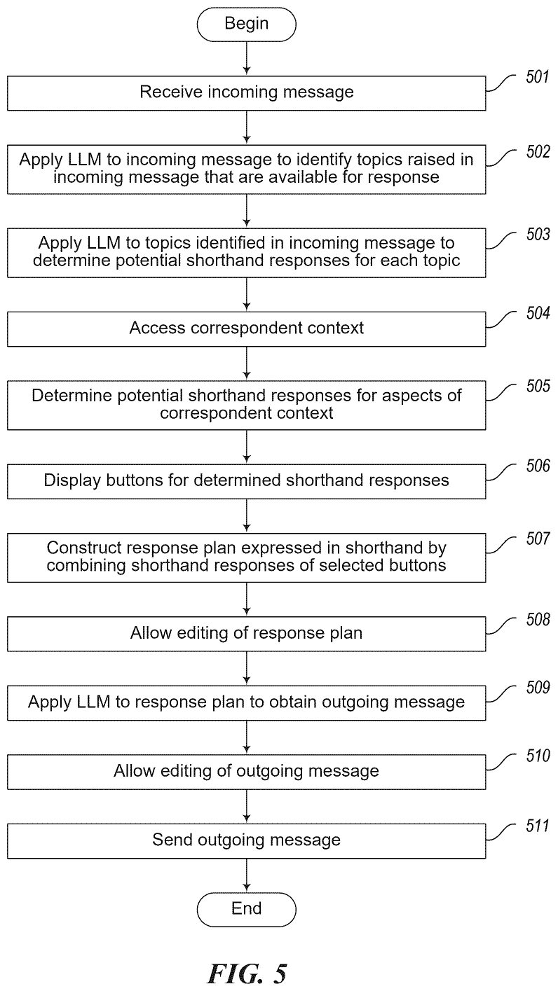

FIG. 5 is a flow diagram showing a process performed by the facility in some embodiments to process an incoming message. In act 501, the facility receives the incoming message. In act 502, the facility applies a large language model to the incoming message received in act 501 to identify topics raised in the incoming message that are available for response. In act 503, the facility applies a LLM to topics identified in act 502 in the incoming message to determine potential shorthand responses for each topic; i.e., candidate response themes for each topic expressed in compact shorthand.

In some embodiments, the facility performs acts 502 and 503 in a single invocation of an LLM. In various embodiments, the LLM used is a version of the OpenAI Generative Pre-Trained Transformer, or “GPT,” such as GPT-4, GPT-4o, or GPT-5. In various embodiments, the facility uses various other LLMs, such as a version of Large Language Model Meta AI (“LLaMA”), such as LLaMA-3, or a version of Anthropic Claude, such as Claude-3.

In some embodiments, in order to perform acts 502 and 530, the facility submits a prompt such as the prompt shown below in Table 1, into which the facility substitutes the incoming message's subject and body in line 48.

TABLE 1
LLM Prompt to Identify Topics and Candidate Response Strategies

| | |
|---|---|
| 1 | { |
| 2 | ″suggestions″: { |
| 3 | ″messages″: [ |
| 4 | { |
| 5 | ″role″: ″system″, |
| 6 | ″content″: ″You are a Medical Assistant. I will provide |
| 7 | you a message from a patient to their physician. Create a list of |
| 8 | questions the patient has for their physician in the message, |
| 9 | remembering the sentence they came from exactly. Then provide one |
| 10 | to three distinct options the physician can choose. Start from the |
| 11 | start of the message and generate a list as a JSON formatted array |
| 12 | of objects including the distinct topic name, the sentence that |
| 13 | should be highlighted for the physician without rephrasing, and an |
| 14 | array of suggestions the physician can choose from - sorted in the |
| 15 | same order the highlighted sentences appear in the message. |
| 16 | Provide the formatted JSON array with the keys: ‘topic‘, |
| 17 | ‘sentence‘ and ‘suggestions‘.″ |
| 18 | }, |
| 19 | { |
| 20 | ″role″: ″user″, |
| 21 | ″content″: ″PATIENT MESSAGE:\n SUBJECT:\nRash\n |
| 22 | BODY:\nDr. Smith, \nI have been having a rash on my leg lately. |
| 23 | It's quite itchy. Should I take benadryl? Should I take the oral |
| 24 | or topical? Can you prescribe a stronger steroid cream? What else |
| 25 | can I do? By the way, I recently started eating vegan. I also need |
| 26 | a refill on my metoprolol.″ |
| 27 | }, |
| 28 | { |
| 29 | ″role″: ″assistant″, |
| 30 | ″content″: ″[{\″topic\″:\″steroid |
| 31 | cream\″,\″sentence\″:\″Can you prescribe a stronger steroid |
| 32 | cream?\″,\″suggestions\″:[\″prescribe\″,\″won't |
| 33 | help\″]},{\″topic\″:\″benadryl\″,\″sentence\″:\″Should I take |
| 34 | benadryl?\″,\″suggestions\″:[\″ok to take\″,\″don't |
| 35 | take\″]},{\″topic\″:\″vegan\″,\″sentence\″:\″By the way, I |
| 36 | recently started eating vegan\″,\″suggestions\″:[\″ok\″,\″good |
| 37 | idea\″]},{\″topic\″:\″refill metoprolol\″,\″sentence\″:\″I also |
| 38 | need a refill on my metoprolol.\″,\″suggestions\″:[\″will |
| 39 | refill\″,\″need visit before refill\″]}]″ |
| 40 | }, |
| 41 | { |
| 42 | ″role″: ″user″, |
| 43 | ″content″: ″SUBJECT:\n{subject}\n BODY:\n{body}″ |
| 44 | } |
| 45 | ], |
| 46 | ″temperature″: 0.0, |
| 47 | ″top_p″: 0.95 |
| 48 | } |
| 49 | } |

The sample prompt above uses a single-shot training technique, according to which the prompt provides a single example of a typical input, and the content and organization of proper output for that sample input. A system portion of the prompt in lines 5-17 gives explicit directions for LLM's generation of output for the input of the incoming message. Lines 19-40 contain the example, of which lines 19-27 contain the example input, and lines 28-40 the example output. It can be seen that the example output is organized as a list of topics, each having the components “topic”, “sentence”, and “suggestions”. The prompt further includes the subject input to be acted on by the LLM in lines 41-44, specifying both the subject and the body of the incoming message for which the LLM is to provide topics, sentences, and suggestions. The prompt further includes a temperature parameter on line 46 indicating a level of randomness to be used by the LLM in generating its response, and a top_p parameter specifying an extent to which less likely words are considered for inclusion in the result. Here, the temperature parameter value of 0.0 is at its lowest level, specifying that the lowest level of randomness is to be used, and the top_p parameter value of 0.95 is near its highest level, specifying less likely words are to be considered to large extent.

In act 504, the facility accesses context for the correspondent, meaning information relating to the state of the relationship between the sender of the incoming message and its addressee. In the case of a message sent by a patient to the patent's physician, in some embodiments, the context relates to recent events in the physician's treatment of the patient, which can be retrieved from the patent's EHR record. In some embodiments, the facility retrieves the contents of recent notes and orders and medications made for the patient by the physician, such as via a HL7 FHIR (Fast Healthcare Interoperability Resources 1) interface exposed by the EHR. In some embodiments, this involves invoking (1) a ServiceRequest FHIR function against the EHR, and (2) a MedicationRequest function.

In act 505, the facility determines potential shorthand responses for aspects of the correspondent context access in act 504. In act 506 the facility displays buttons or other controls for the shorthand responses determined in act 503 (e.g., buttons 431-433 and 441-442) and shorthand responses determined in act 505 (e.g., buttons 461 and 471-479). In act 507, the facility constructs a response plan (such as response plan 480) that is expressed in shorthand by combining the shorthand responses of the buttons displayed in act 506 that were selected by the user—in the example, buttons 431, 461, 475, and 479. In act 508, the facility allows editing of the response plan constructed in act 507.

In act 509, the facility applies an LLM to the response plan constructed in act 507 and edited—if at all—in act 508 to obtain an outgoing message. In some embodiments, in act 509, the facility invokes an LLM using the prompt shown below in Table 2, substituting in lines 41-42 to substitute the subject and body of the incoming message as well as the response plan, as edited. In various embodiments, the facility submits this prompt to the same LLM as the prompt shown in Table 1, or to a different LLM.

TABLE 2
LLM Prompt to Generate Outgoing Message

| | |
|---|---|
| 1 | { |
| 2 | “reply”: { |
| 3 | “messages”: [ |
| 4 | { |
| 5 | “role”: “system”, |
| 6 | “content”: “You are a Medical Assistant. Your job is to |
| 7 | read a PATIENT MESSAGE to their physician, as well as a brief |
| 8 | shorthand RESPONSE written by their doctor and combine them to |
| 9 | create a response to the patient. “ |
| 10 | }, |
| 11 | { |
| 12 | “role”: “user”, |
| 13 | “content”: “RESPONSE\nask fever, discharge, blisters. will |
| 14 | send steroid rx. followup no improvement 1 week\n\nPATIENT |
| 15 | MESSAGE:\n SUBJECT:\nRash\n BODY:\nDr. Smith,\nI have |
| 16 | been having a rash on my leg lately. It's quite itchy. What can I |
| 17 | do?” |
| 18 | }, |
| 19 | { |
| 20 | “role”: “assistant”, |
| 21 | “content”: “Can you tell me if there has been any |
| 22 | discharge or blistering? Have you had a fever? I would guess this |
| 23 | will improve with some steroids, I'll write you a prescription. |
| 24 | Follow up with me in a week if things don't improve. I know rashes |
| 25 | can be really frustrating. I hope you feel better soon! Let me |
| 26 | know if you have any questions.” |
| 27 | }, |
| 28 | { |
| 29 | “role”: “user”, |
| 30 | “content”: “RESPONSE\n{plan}\n\nPATIENT |
| | MESSAGE:\n |
| 31 | SUBJECT:\n{subject}\n BODY:\n{body}” |
| 32 | } |
| 33 | ], |
| 34 | “temperature”: 0.3, |
| 35 | “top_p”: 0.7 |
| 36 | } |

The prompt shown in Table 2 similarly uses the single-shot training technique, including explicit directions in the system portion of the prompt on lines 4-10; example input on lines 11-18; example output on lines 19-27; and input to be processed on lines 28-32. In lines 34 and 35, the prompt specifies a moderately low temperature of 0.3 out of 1.0, and a moderately high top_p value of 0.7 out of 1.0.

In act 510, the facility permits the user to edit the outgoing message obtained in act 509. In act 511, once any editing of the outgoing message is complete, the facility sends it to the user who sent the incoming message. After act 511, this process concludes.

Those skilled in the art will appreciate that the acts shown in FIG. 5 may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method in a computing system, comprising:
receiving an incoming textual message received from a sending address;
invoking a first large language model against the incoming textual message at least once to (1) identify in the incoming textual message one or more topics for which response would be appropriate and (2) for each of the identified topics, generating a plurality of candidate response themes;
for each of the identified topics, causing to be displayed a control visually identifying each of the generated candidate response themes;
receiving input selecting a subset of the displayed controls;
collecting shorthand for the candidate response themes of the selected message into a response plan;
causing the response plan to be displayed;
receiving input adjusting the shorthand in the displayed response plan;
invoking a second large language model against the response plan in which the shorthand was adjusted to generate an outgoing message; and
causing the generated outgoing message to be transmitted to the sending address.

2. The method of claim 1 wherein the first large language model is the same as the second large language model.

3. The method of claim 1, further comprising causing the incoming textual message to be displayed simultaneously with the controls.

4. The method of claim 1 wherein each of the displayed controls contains the shorthand for the candidate response team visually identified by the control.

5. The method of claim 1, further comprising:
causing the generated outgoing message to be displayed; and
receiving input adjusting the displayed outgoing message,
wherein it is the adjusted outgoing message that is transmitted to the sending address.

6. One or more memories collectively storing a data structure, the data structure comprising:
first information configured to cause display of an incoming message; and
for each of one or more topics automatically identified in the incoming message:
for each of one or more candidate response themes automatically generated for the topic:
second information configured to cause display of a control bearing a shorthand characterization of the candidate response theme,
such that, upon the caused display, the controls are selectable to specify the corresponding candidate response themes.

7. The one or more memories of claim 6, the data structure further comprising third information configured to cause the controls of a subset of the candidate response themes to be displayed in a way that is visually distinct from the controls not included in the subset to reflect user selection of the controls of the subset.

8. The one or more memories of claim 7, the data structure further comprising fourth information configured to cause display of a response plan automatically generated by combining the shorthand characterizations of the candidate response themes whose controls are among the subset.

9. The one or more memories of claim 8 wherein the displayed response plan reflects user revisions.

10. The one or more memories of claim 8, the data structure further comprising fifth information configured to cause display of an outgoing message automatically generated in accordance with the response plan.

11. The one or more memories of claim 10 wherein the displayed outgoing message reflects user revisions.

12. The one or more memories of claim 10, the data structure further comprising fifth information configured to cause display of a control that can be activated to cause the sending of the outgoing message to an originator of the incoming message.

13. The one or more memories of claim 6, the data structure further comprising third information configured to cause display of one or more controls each bearing a shorthand characterization of the candidate context theme reflecting contextual information for an originator of the incoming message.

14. One or more memories collectively having contents configured to cause a computing system to perform a method, the method comprising:
receiving an incoming textual message received from a sender;
invoking a first large language model against the incoming textual message at least once to (1) identify in the incoming textual message one or more topics for which response would be appropriate and (2) for each of the identified topics, generating a plurality of candidate response themes; and
for each of the identified topics, causing to be displayed controls visually identifying each of the generated candidate response themes.

15. The one or more memories of claim 14, the method further comprising:
retrieving one or more items of context information for the sender; and
for each of the retrieved items of context information, causing to be displayed a control visually identifying the retrieved item of context information.

16. The one or more memories of claim 15 wherein the retrieving retrieves the one or more items of context information from a record of an electronic health record system maintained for the sender.

17. The one or more memories of claim 14, the method further comprising:
receiving input selecting a subset of the displayed controls;
collecting shorthand for the candidate response themes of the selected message into a response plan; and
causing the response plan to be displayed.

18. The one or more memories of claim 17, the method further comprising:
invoking a second large language model against the response plan in which the shorthand was adjusted to generate an outgoing message; and
causing the generated outgoing message to be displayed.

19. The one or more memories of claim 18, further comprising:
before invoking the second large language model, receiving input revising the displayed response plan to be displayed,
and wherein it is the revised response plan against which the second large language model is invoked.

20. The one or more memories of claim 18, further comprising causing the generated outgoing message to be transmitted to the sender.

21. The one or more memories of claim 18, the method further comprising:
before causing the generated outgoing message to be transmitted, receiving input revising the displayed outgoing message,
and wherein it is the revised outgoing message that is transmitted to the sender.
